(19) United States
(12) Patent Application Publication
(10) Pub. No.: US 2004/0259144 A1
(43) Pub. Date: Dec. 23, 2004

(54) Method for diagnosing and treating breast cancer

(76) Inventor: Nagabhushana Prabhu, West Lafayette, IN (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/855,991

(22) Filed: May 27, 2004

Related U.S. Application Data
(60) Provisional application No. 60/473,414, filed on May 27, 2003.

(51) Int. Cl. C12Q 1/68; G01N 33/574; G06F 19/00; G01N 33/48; G01N 33/50

(52) U.S. Cl. 435/6; 702/20; 435/7.23

(57) ABSTRACT

A method of evaluating progress of a cancer comprises calculating a prognostic index by selecting coefficients for each of a plurality of nuclear pleomorphic features using a numerical regression, such that the selected coefficients provide a correlation extremum to times to recurrence. A prognostic index is calculated by measuring nuclear pleomorphic features in a patient, and multiplying the observed values by the corresponding selected coefficients. The resulting prognostic index can provide a superior prediction of the likely time to recurrence in the patient.

### CLAIM OF PRIORITY

This application claims priority from U.S. Provisional Patent Application No. 60/473,414, filed May 27, 2003, which is hereby incorporated by reference.

### BACKGROUND

Predicting the progress of cancer is important for helping doctors and patients to make informed decisions about the best treatment options. The more accurately the progress of the disease can be predicted, the better able patients are to receive effective treatment with the least side effects.

Typically, after a patient is found to have cancer, the extent of spread of the disease is estimated, using the “TNM staging system.” TNM staging classifies the tumor into four stages, based on the tumor size, lymph node invasion and the metastatic status. Stage I corresponds to the disease in its early stages, while stage IV corresponds to an advanced stage of the disease (with evidence of distant metastatic secondary growths). This conventional staging gives an estimate of spread of the tumor. However, in order to make well-informed treatment decisions, the chance of recurrence must be assessed. The assessment of the chance of recurrence relies on “prognostic indices.”

Prognostic indices are numbers that are calculated from measurable features, that have been shown to be related to the historical rates of cancer recurrence. Historical rates of recurrence are measured using the “time to recurrence” (“TTR”). This metric refers to the amount of time after a diagnosis until cancer recurred in the patient. The usefulness of a prognostic index is related to the degree of correlation between the index and the observed TTR—the further the correlation is from zero (either positive or negative), the more helpful the prognostic index.

Breast cancer is one of the most damaging kinds of cancer today. Excluding skin cancer, breast cancer is the most common cancer in American women. It fact, roughly one in three cancers diagnosed each year is a breast cancer. Breast cancer ranks behind only lung cancer as a cause of cancer-related deaths. The American Cancer Society predicted that approximately 205,000 new invasive cases of breast cancer and 40,000 deaths due to breast cancer occurred in the United States in 2002.

The progress of breast cancer is frequently predicted using the Nottingham Prognostic Index (“NPI”), especially in Europe. The NPI is based on the tumor size, the lymph node status and the histologic grade. The histologic grade, in turn, is typically determined using the modified Scarff-Bloom-Richardson (SBR) system, which is based on an assessment of the extent of microtubule formation, mitotic index, and nuclear pleomorphism. The SBR system is also frequently used as a stand-alone prognostic index, especially in the U.S. Thus, two of the most common methods of evaluating the progress of cancer rely upon measurement of nuclear pleomorphism.

“Nuclear pleomorphism” refers to the development of cell nuclei with a variety of forms. The types of features that pleomorphism observes include, for example, the radius, texture, symmetry, smoothness, and other such morphological features. Typically, it is evaluated by visual inspection of a tissue sample under a microscope. Although this diagnostic is widely used, it is inherently subjective. Different doctors can examine the same sample, and reach different conclusions about the extent of pleomorphism. This limits the clinical value of the tumor grade for predicting the chance of or time to recurrence.

Because of the importance of nuclear pleomorphism to common methods of evaluating cancer, this problem has been studied extensively. Research has been done to try to automate the determination of a tumor's nuclear grade—so far, without a satisfactory solution.

Those skilled in the art will appreciate that the biological basis for the prognostic significance of the nuclear pleomorphism is slowly beginning to emerge, through recent research on cytoskeleton and nuclear matrix. Acquisition of the metastatic potential requires alteration of the cell's motility and adhesion properties, both of which are linked to the cytoskeleton. Recent studies report a correlation between altered regulation of the cytoskeleton and the progression to metastatic phenotype. For example, studies have shown that a point mutation in Fibronectin, an extracellular glycoprotein that regulates cytoskeletal organization, confers metastatic potential on tumor cells. Also, synthetic polypeptides that mimic the active site of Fibronectin are shown to inhibit metastasis, lending further evidence to the role of fibronectin in metastasis.

Recent findings also show that the enhanced expression of RhoC, a member of the Rho GTPase family which regulates cytoskeletal organization, correlates with the progression of pancreatic adenocarcinoma and melanoma to the metastatic phenotype. In addition, the altered expression of Thymosin β4, a protein that regulates actin polymerization, and other members of its family, namely Thymosin β10 and Thymosin β15, are also found to be correlated with the onset of metastasis. Other cytoskeletal regulator proteins, whose expression levels are found to be correlated with metastasis, include α-catenin, α-actinin and α-centractin.

Altered cytoskeletal regulation, which appears to be an important event in a tumor's progression to metastatic phenotype, also appears to be coupled to the alterations in the nuclear matrix which, like the cytoskeleton for a cell, provides the structural scaffold for the nucleus. For example, actin, a major component of the microfilament network of the cytoskeleton, has also been identified within the nuclear matrix and may provide an organizational scaffold for a variety of intranuclear processes including chromatin remodeling to splicing. The correlation between the onset of metastasis and the altered expression of Thymosin β4, a protein that regulates actin polymerization, suggests that alongside the cytoskeleton, the altered actin polymerization, plausibly, also affects the nuclear matrix and hence the nuclear shape and texture. In addition, the altered activity of malignancy-specific nuclear matrix protein p114 has been reported to occur only in human breast carcinomas and not in normal breast tissues. Studies on rat and human prostate tumors have also a correlation between changes in nuclear matrix composition with malignancy. Besides the above-mentioned reports the role of nuclear matrix proteins in the transformation of a tumor cell to metastatic phenotype has also been reported in bladder cancers, colon cancers, breast cancers and the squamous cell carcinomas of the head and the neck.

Thus, research is beginning to show a correlation between the alterations of a cell's cytoskeleton and nuclear matrix on the one hand and the cell's progression to metastatic phenotype on the other. Our current understanding of the molecular processes occurring on the nuclear matrix scaffold is inadequate to determine if the alterations in the nuclear matrix is a cause or a consequence of the acquisition of metastatic potential. However, it is becoming increasingly clear that the alterations in the nuclear matrix, manifested as the alterations in the nuclear morphology, is a critical marker of the virulence of the disease. Hence the quantitative nuclear pleomorphism indices described hereinbelow can be valuable in predicting the clinical progress of the disease.

The correlations between the clinical progress of tumors and individual nuclear morphological features such as mean nuclear area have also been studied. However, individual features, such as mean nuclear area, provide only a partial description of nuclear pleomorphism.

Thus, what is needed is an improved method for predicting and evaluating the progress of cancers. Methods that produce more consistent results when performed by different practitioners are also needed. Improved methods that can be used to evaluate and predict the progress of breast cancer are needed. Methods of doing these things that employ the relationship between metastasis and changes in cellular nuclei are needed. The present invention is directed to meeting these needs, among others.

### SUMMARY OF THE INVENTION

The present invention provides means to overcome the problems associated with low inter-observer reproducibility and improve the reliability of the prognosis in evaluation of cancer.

In one aspect of the invention, improved evaluation of cancer is provided through a composite quantitative measure of nuclear pleomorphism, referred to herein as the nuclear pleomorphism index (“NPI”). The NPI depends only on measurable morphological nuclear features. In the presently preferred embodiment, the relative weights on the features are determined using a constrained nonlinear optimization procedure.

In a second aspect of the invention, improved evaluation of cancer is provided through a composite quantitative measure, referred to herein as the composite prognostic index (“CPI”). In the presently preferred embodiment, the CPI includes measurable morphological nuclear features, and two other measurable tumor features: tumor size and lymph node status.

In a third aspect of the invention, improved evaluation of cancer is provided by methods that employ the newly discovered, strong relationship between TTR and nuclear compactness, texture, or both. In certain embodiments, such methods employ measurements of these factors that are more precise, more accurate, or both, than the measurements employed in prior art evaluation and diagnosis methods.

In a fourth aspect of the present invention, a single composite index is computed by combining the values of several nuclear morphological features, which provides a quantitative measure of nuclear pleomorphism correlating highly with the clinical progress of the tumor.

### BRIEF DESCRIPTION OF THE DRAWINGS

Although the characteristic features of this invention will be particularly pointed out in the claims, the invention itself, and the manner in which it may be made and used, may be better understood by referring to the following descriptions taken in connection with the accompanying figures forming a part hereof.

FIG. 1 is a graph illustrating the correlations between TTR and 32 tumor features.

FIG. 2 is a graph illustrating an optimal set of feature weights for an NPI according to the present invention, based on the data in the Wisconsin Breast Cancer Database.

FIG. 3 is a graph illustrating an optimal set of feature weights for a CPI according to the present invention, based on the data in the Wisconsin Breast Cancer Database.

### DETAILED DESCRIPTION OF THE PREFERRED EMBODIMENTS

For the purposes of promoting an understanding of the principles of the invention, the preferred embodiment of the invention and certain alternative embodiments deemed helpful to further explain the invention will now be described. Although the language used is specific to those embodiments, it will nevertheless be understood that the scope of the invention is not meant to be thereby limited. Those skilled in the art will recognize various alternations and further modifications to the embodiments discussed herein. These further applications of the principles of the invention are contemplated, and desired to be protected. However, it is not feasible to describe every possible embodiment of the invention, nor is it necessary to do so in order to adequately describe the invention and to enable those skilled in the art to make use of it. As a particular example, while the presently preferred embodiment relates to the evaluation and prediction of the progress of breast cancer, those skilled in the art will appreciate that the methods according to the present invention can also be applied to other kinds of cancer.

An improved method of evaluating and diagnosing cancer according to the present invention provides better information to doctors and patients about the progress of the disease. This information helps doctors and patients to select the best treatment options, based on the patient's individual circumstances. Patients will, therefore, be more likely to receive effective treatment, with less troublesome side effects.

In particular, the correlation between time to recurrence (“TTR”) and the nuclear pleomorphism index (“NPI”) calculated from the Wisconsin Breast Cancer Database, as described below, was found to be 29.21% (32.33%) better than the correlation between TTR and any single feature. The correlation between TTR and the composite prognostic index (“CPI”) was found to be 32.33% better than the correlation between TTR and any single feature.

Furthermore, NPI and CPI provide more accurate evaluation of the progress of cancer, because they resolve the inter-observer reproducibility problem—that is, because they provide more consistent results, even when performed by different practitioners.

Construction of the NPI and CPI can be illustrated using the digital morphological data in the Wisconsin Breast Cancer Database (“WBCD”). The WBCD contains the data obtained from the fine needle aspirates of 198 invasive ductal carcinomas (showing no distant metastases at the time of diagnosis). For each case, the mean, standard deviation and extreme (largest) values of each of ten morphological features have been measured using an interactive software package. The ten morphological features are: (1) radius; (2) texture; (3) perimeter; (4) area; (5) smoothness; (6) compactness; (7) concavity; (8) concave points; (9) symmetry; and (10) fractal dimension. In addition to these 30 values, for each case, the database also contains a measurement of the tumor size, lymph node status and the TTR. For the cases in which the disease recurred the TTR was recorded as the interval between diagnosis and recurrence and in the cases in which the disease did not recur, the TTR field represents the disease-free survival time. Thus, for each case, the database contains 33 numerical values as described above. The mean, standard deviation, and extreme values of the 10 nuclear features, together with the lymph node status and the tumor size form a 32-dimensional vector.

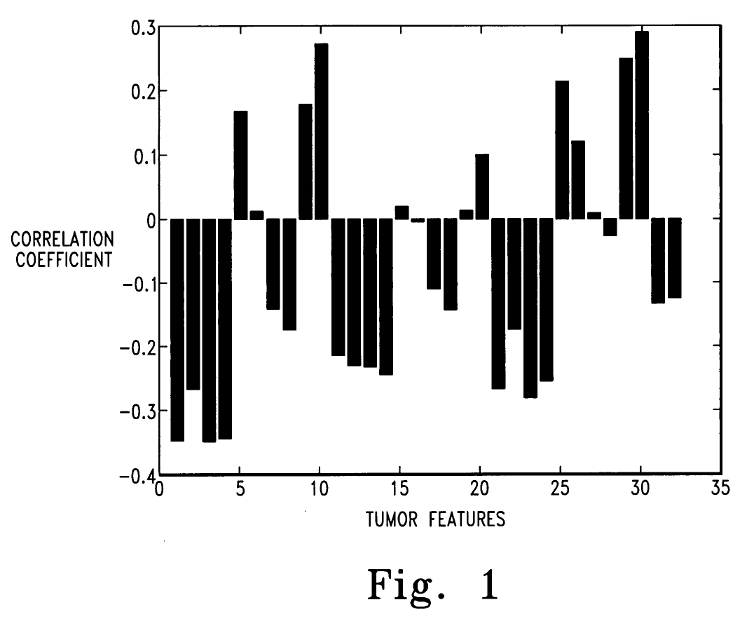

FIG. 1 illustrates the correlation between each of these 32 tumor features and the TTR across the 198 cases in the WBCD.

From this data, an NPI can be constructed. This can be done by performing a nonlinear regression to determine the optimal combination of the 30 nuclear morphological features, which yields the largest anti-correlation with the TTR, as follows:

(1) Let V₁, . . . , V₁₉₈ denote the 30-dimensional vectors corresponding to the nuclear features of the 198 cases. Let β=(β₁, . . . , β₃₀) be the vector of coefficients for a particular combination of feature values.

(2) Solve the following nonlinear optimization problem,

$$Minimize \frac{{{\sum }_{i = 1}^{}}^{198} \{ ( ©β , {V}_{i}^{} - v ) ( {T}_{i} - τ ) \}}{{{[ {\sum }_{i} {( ©β , {V}_{i}^{} - v )}^{2} ]}^{1 / 2} [ {\sum }_{i} {( {T}_{i} - τ )}^{2} ]}^{1 / 2}}$$

$$Such that \text{:}$$

$${\sum }_{i} ©β , {V}_{i}^{} = 198 v$$

$${\sum }_{i} {T}_{i} = 198 τ$$

The optimal solution β* yields the relative weights for the 30 tumor features. The combination of the tumor features obtained by weighting the features using β* yields the optimal NPI (based on the data set used).

The optimization problem can be solved, for example, using the fminunc optimizer in MATLAB. It should be noted that the objective function could have several local minima. Since solving the optimization problem this way will locate a local minima about the initial β value, the optimization problem should be solved multiple times, using a variety of initial β values. The best solution among the computations should be chosen as the optimal β*. It will be appreciated by those skilled in the art that any suitable method of solving the minimization problem may be used.

The above optimization provides an NPI. Similar computations can be performed to construct a CPI. This is done by combining the 30 nuclear features with the tumor size, the lymph node state, or both. If both the tumor size and lymph node factor are used, the following optimization problem results:

$$Minimize \frac{{{\sum }_{i = 1}^{}}^{198} \{ ( ©α , {W}_{i}^{} - λ ) ( {T}_{i} - τ ) \}}{{{[ {\sum }_{i} {( ©α , {W}_{i}^{} - λ )}^{2} ]}^{1 / 2} [ {\sum }_{i} {( {T}_{i} - τ )}^{2} ]}^{1 / 2}}$$

$$Such that \text{:}$$

$${\sum }_{i} ©α , {W}_{i}^{} = 198 λ$$

$${\sum }_{i} {T}_{i} = 198 τ$$

It will be appreciated that W₁ . . . W₁₉₈ are 32-dimensional vectors obtained by adding to V₁, . . . , V₁₉₈ two extra coordinates corresponding to the lymph node status and the tumor size. A 32-dimensional coefficients vector, α, replaces the β in the previous problem. As before, to account for the possibility of multiple local minima, the problem should be solved numerous times, with a variety of initial α. The best solution among the computations should be chosen as α*.

FIG. 1 illustrates the correlation coefficient of each of the 32 tumor features and the TTR that was found running the optimization problem 100 times, using random initial values. The minimum correlation between TTR and a single nuclear feature is −0.3461, which is attained by the mean nuclear perimeter. The anti-correlations of the mean nuclear radius and the mean nuclear area are nearly as high (−0.3447 and −0.3440 respectively).

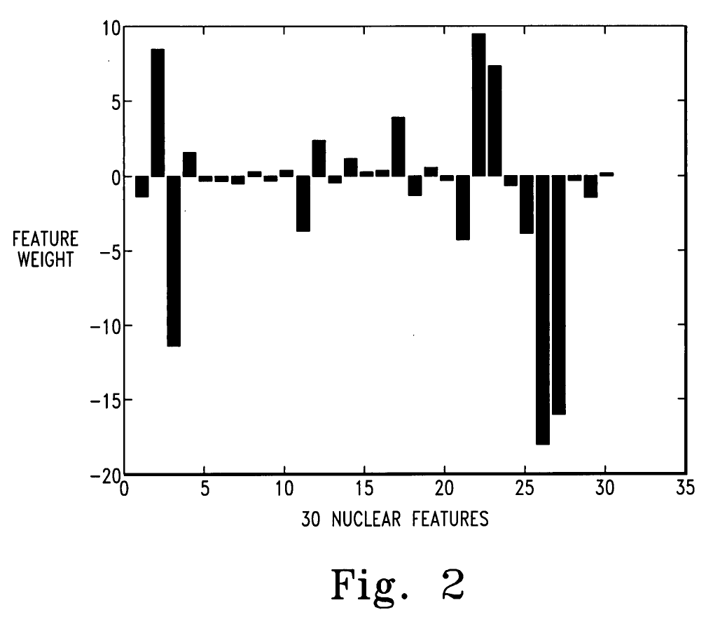

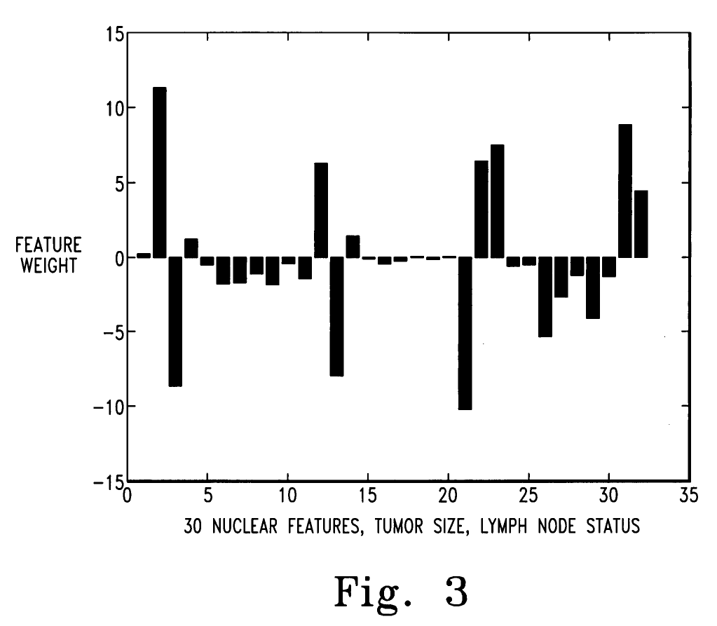

FIGS. 2 and 3 show the optimal solutions for the NPI and CPI. Note that the scales of the coefficients shown in FIGS. 2 and 3 are not significant, since the correlation coefficient is a scale-invariant measure. The figures use rescaled relative weights selected to make the absolute values of the relative weights add up to 100. Thus each relative weight, regardless of its sign, can be interpreted as a relative percentage significance of the corresponding feature.

The precise numerical values of the feature weights in FIGS. 2 and 3 are presented in the table below:

TABLE 1
Optimal weights for the 30 tumor features in FIG. 2:

| | Mean | Standard Deviation | Extrema |
|---|---|---|---|
| Radius: | −1.7049 | −4.9436 | −5.7212 |
| Texture: | 11.4098 | 3.0582 | 12.7601 |
| Perimeter: | −15.3830 | −0.6380 | 9.8179 |
| Area: | 1.9327 | 1.5116 | −0.8603 |
| Smoothness: | −0.4017 | 0.2813 | −5.3482 |
| Compactness: | −0.3503 | 0.4984 | −24.2981 |
| Concavity: | −0.6530 | 5.1504 | −21.6803 |
| Concave Points: | 0.2198 | −1.7866 | −0.3255 |
| Symmetry: | −0.3638 | 0.7240 | −1.8973 |
| Fractal Dimension: | 0.3753 | −0.4352 | 0.0609 |

TABLE 2
Optimal weights for the 32 tumor features in FIG. 3:

| | | |
|---|---|---|
| Mean | Standard Deviation | Extrema |

Radius:
Texture:
Perimeter:
Area:
Smoothness:
Compactness:
Concavity:
Concave Points:
Symmetry:
Fractal Dimension:
Tumor Size:
Lymph Node:

The correlation between TTR and the NPI is −0.4472 (FIG. 2), which represents a 29.21% improvement over the highest correlation (−0.3461) between TTR and any single nuclear feature. Considering the tumor size and lymph node status in addition to the nuclear features, the correlation between the CPI and TTR improves further to −0.4580, which is a 32.33% improvement over the best single-feature correlation. Thus, the NPI and CPI have significantly higher correlations with TTR, than any single nuclear feature, tumor size or lymph node status.

The single-feature correlations with TTR, shown in FIG. 1, are consistent with the results in the relevant literature. The mean nuclear size, captured in the WBCD data in the radius, perimeter, and area, has a better anti-correlation with TTR than either the tumor size or the lymph node status. The mean perimeter emerges as the feature having the highest anti-correlation (−0.3461) with TTR, although the single-feature anti-correlation is significantly lower than what either of the composite indices provide.

The results shown in FIGS. 2 and 3 should be interpreted as follows. The relative weights represent the sensitivity of the composite index to the change in the corresponding feature value. For example, as shown in FIG. 2, the NPI has a correlation coefficient of −0.4472 with TTR, implying that, statistically, as NPI increases, the TTR decreases, and vice versa. FIG. 2 shows that among all the features, the extreme value of nuclear compactness has the largest relative weight in NPI (−18.05%). In contrast, the mean nuclear radius has a relative weight of 1.27%. Recall that NPI is defined as

NPI=Σ(value of i^{th} feature)*(relative weight of i^{th} feature); 1<i<30

Therefore, an increase in the extreme value of the nuclear compactness by an amount Δ, decreases the NPI by an amount equal to −18.05Δ, whereas an increase in the mean nuclear radius by the same amount Δ, leads to only a decrease of −1.27Δ, in the NPI. Thus, NPI is about 14.21 times more sensitive to changes in the extreme value of the nuclear compactness than it is to changes in the mean nuclear area. Stated differently, a nearly 14-fold smaller change in the extreme value of nuclear compactness affects the NPI to the same extent as a change in the mean nuclear area. Hence, in summary, NPI is found to be most sensitive to changes in the extreme value of nuclear compactness.

Nuclear compactness is the dimensionless number, computed as follows:

$$Compactness = \frac{{( Perimeter )}^{2}}{Area} .$$

The isoperimetric inequality of plane geometry states that all the closed (rectifiable) curves (in the 2-dimensional plane) having length L and enclosing area A, satisfy the following isoperimetric inequality:

L²−4πA≧0

Further, the inequality is satisfied as equality only by the circle. In other words, among all possible closed curves, the circle has the lowest perimeter for a given area, and hence is most compact, according to the above definition of compactness. Therefore, increase in the compactness is a measure of deviation from circularity.

It will be appreciated that the same problems can be solved for the largest correlation, rather than for the largest anti-correlation, to provide essentially the same information. The resulting data is simply interpreted in the opposite way, in order to account for the sign change. In other words, a positive correlation between NPI or CPI and TTR means that a higher NPI or CPI predicts a longer time to recurrence.

The high sensitivity of the NPI calculation described above to nuclear compactness supports the postulated correlation between nuclear matrix disruption and the onset of metastasis. Thus, based on these calculations, the data in the WBCD suggest that changes in the extreme nuclear compactness characteristic represent a morphological manifestation of the breakdown of nuclear matrix organization. As previously discussed, there is increasing evidence that the disruption of the nuclear matrix scaffold is strongly correlated to, and possibly precedes, the onset of metastasis. Therefore it is plausible to expect that the progression to aggressive metastatic phenotype is correlated to the expression of extreme values of nuclear compactness.

It will be appreciated that, in light of the large sensitivity of TTR to changes in nuclear compactness, the accuracy of prediction of TTR is greatly enhanced through accurate measurement of this feature. One means of achieving that enhanced accuracy would be to use automated feature extraction schemes.

The interpretation of relative weights, presented above, can be used to analyze the other results shown in FIGS. 2 and 3. Thus, while the NPI has the highest sensitivity to extreme nuclear compactness, the CPI, which includes tumor size and lymph node status, has the highest sensitivity to mean texture. Therefore, increase in the mean texture value leads to greatest increase of CPI, with a corresponding reduction in TTR.

In summary, the relative feature weights, computed using nonlinear optimization, are shown to improve the correlation with TTR significantly, and also expose the high sensitivity of TTR on nuclear compactness and texture. These two findings are expected to provide valuable clinical guidance to the prediction of TTR of breast carcinomas.

While the calculations described herein employed the data in the WBCD, it will be appreciated that corresponding calculations can be performed on any suitable data. As will be familiar to those skilled in the art, all other things being equal, the predictive value of calculations based on such data will often be improved by increasing the size of the data set, and by improving the accuracy of the measurements included in the data set.

It will also be appreciated that, NPIs and CPIs calculated from alternative data sets may show different relative sensitivities to the parameters discussed herein. For example, NPIs calculated from other databases might be most sensitive to mean texture, as was the CPI in the calculations based on the WBCD.

Those skilled in the art will also appreciate that NPIs and CPIs can be calculated from data sets that include additional parameters, or which lack some of the parameters included in the WBCD. An NPI according to the present invention can be calculated using any set of data including morphological data from which a quantitative measure of nuclear pleomorphism can be extracted. Likewise, a CPI according to the present invention can be calculated using any set of data from which an NPI can be calculated, so long as it also includes corresponsing data on tumor size, lymph node status, or both.

It will also be appreciated that prognostic indices according to the present invention can be calculated using linear regressions. Because of the nature of quantitative relationships in biological systems, however, nonlinear regression is presently regarded by the inventors as the preferred means of calculating prognostic indices according to the present invention.

While the invention has been illustrated and described in detail in the drawings and foregoing description, this description is merely illustrative. It is not intended to restrict the scope of the invention, since only the preferred embodiments, and such alternative embodiments deemed helpful in further illuminating the preferred embodiment, have been shown and described. It will be appreciated that changes and modifications to the foregoing can be made without departing from the scope of the following claims.

What is claimed is:

1. A method of evaluating an expected time to recurrence of a breast cancer in a patient, the method comprising the steps of:
(a) providing numerical data for a plurality of observed times to recurrence and an associated plurality of nuclear pleomorphic features comprising: radius; texture; perimeter; area; smoothness; compactness; concavity; concave points; symmetry; and fractal dimension;
(b) performing a nonlinear regression on the numerical data to select a coefficient for each of the plurality of nuclear pleomorphic features, such that the selected coefficients provide a local extreme in the correlation to the corresponding times to recurrence;
(c) measuring at least two of the plurality of nuclear pleomorphic features in tumor tissue of the patient; and
(d) calculating a prognostic index by multiplying each of the at least two measured pleomorphic features in tumor tissue of the patient by the corresponding coefficients selected in step (b).
